(19) United States
(12) Patent Application Publication
(10) Pub. No.: US 2015/0182570 A1
(43) Pub. Date: Jul. 2, 2015

(54) COMPOSITION HAVING A. paniculata FOR WOUND HEALING AND SKIN WHITENING, AND METHOD BY USING THE SAME

(71) Applicant: National Dong Hua University, Hualien (TW)

(72) Inventors: CHING-FENG WENG, Hualien (TW); Yi-Chen Chia, Hualien (TW); Chia-Jen Wu, Hualien (TW); Chih-Chien Tseng, Hualien (TW)

(73) Assignee: National Dong Hua University, Hualien (TW)

(21) Appl. No.: 14/145,475

(22) Filed: Dec. 31, 2013

(51) Int. Cl. A61K 36/19; A61Q 19/02; A61K 8/97; A61K 31/341; A61K 8/49

(52) U.S. Cl. CPC A61K 36/19; A61K 31/341; A61K 8/4973; A61K 8/97; A61Q 19/02

(57) ABSTRACT

The present invention provides a pharmaceutical composition for wound healing and/or skin whitening. The pharmaceutical composition comprises active ingredient of Andrographis paniculata extract and/or Andrographolide and a pharmaceutical acceptable carrier. The present invention also provides a method for wound healing and/or skin whitening by using the present pharmaceutical composition. The composition and method of the present invention show better efficacy in wound healing and skin whitening than conventional drugs.

### BACKGROUND

1. Technical Field

The present disclosure relates to composition and method for wound healing, especially, for composition having Andrographis paniculata derivatives.

2. Description of Related Art

The wound healing of skin is a complicated process that is involved in a variety of growth factors, cytokines, cell types and extracellular matrix (ECM) (Breitkreutz et al, 2009). The process of wound healing is composed of inflammation, granulation tissue proliferation, and epidermal is cell regeneration and remodeling (Takayama & Aoki, 2012). The time points among these processes are not precise defined, and different cells and substances are involved in each process (Eming et al, 2007). The remodeling process is the last and the longest phase of wound healing. Decreased protein synthesis and cell proliferation, but increased collagen deposition organized fibrils were observed during this process. The macrophages, endothelial cells and myofibroblasts undergone apoptosis, or escaped from the wound (Gurtner et al, 2008). The wound remodeling process is a balance between ECM production, breakdown and remodeling. This balance is mediated by infiltrated Th1 or Th2 cells, and maintained by subsequently infiltrated macrophages or other immune cells (Tredget et al, 2006; Zaja-Milatovic & Richmond, 2008). The rate of wound healing depends on the cause of injury and type of wound. The keloid, hypertrophic scar and ulcer are the problems of wound healing (Mogili et al, 2012). Re-epithelialization is an important step for wound healing process and is driven by increased proliferation and migration of keratinocytes to wound area. Proliferation and migration of keratinocytes are stimulated by local wound milieu that typically shows an altered composition of extracellular matrix and the presence of cytokines are produced by cells of granulation tissue and clot (Singer & Clark, 1999).

Andrographis (A.) paniculata is an annual herb, which grows in tropical Asia, Africa and South America. Previous articles mentioned that A. paniculata not only effectively reduced fever (Caceres et al, 1999; Spasov et al, 2004), but also improved the formation of blood clots through modulation of anti-platelet function (Thisoda et al, 2006). Several functions of A. paniculata were also been identified, such as reduce the blood pressure (Yoopan et al, 2007), inhibit cancer cell growth (Jin et al, 2012; Yang et al, 2010), and anti-inflammation (Jin et al, 2012). Andrographolide, Andrographoside, 14-deoxyandrographolide, 14-deoxy-11,12-didehydroandrographolide and flavonoids are main constituents of A. paniculata. Andrographolide is the active compound obtained from the aerial parts of plant and it exhibits multiple pharmacological activities. It has been shown to exhibit hepatoprotective activity in galactosamine induced acute hepatitis in rats (Handa & Sharma, 1990) and useful for the treatment of jaundice (Sharma et al, 2012).

The fact that A. paniculata has potentials in various pharmaceutical applications draws demands and interests in the art to evaluate more pharmaceutical applications of A. paniculata so that the economic value thereof can be even increased.

### SUMMARY

One of the objects of the present invention is to increase the economic value of A. paniculata by evaluating its pharmaceutical applications.

Another object of the present invention is to provide a novel pharmaceutical composition and method for wound healing and/or skin whitening, preferably by using A. paniculata.

In order to achieve the above-mentioned objects, the present invention provides a pharmaceutical composition for wound healing and/or to skin whitening, comprising: 0.01 to 1 wt % of an active ingredient, comprising an Andrographis paniculata extract, Andrographolide, or a combination thereof; and 90 to 99.99 wt % of a pharmaceutical acceptable carrier.

Preferably, said pharmaceutical acceptable carrier is water, alcohol, glycerol, chitosan, alginate, chondroitin, Vitamin E, Vitamin A, mineral oil, dimethyl sulfoxide (DMSO), or a combination thereof.

Preferably, an administrating route of said composition via spreading said composition on a place to be treated of an object.

Preferably, said Andrographis paniculata extract is made by the following steps: obtaining an Andrographis paniculata plant; soaking said Andrographis paniculata with an ethanol to obtain a mixture; concentrating said mixture to form said extract.

Preferably, said soaking is performed at 20 to 25° C. for 90 to 96 hours.

Preferably, said extraction process further comprises the step of dissolving said extract in DMSO, absolute ethanol, anhydrous ethanol, or a combination thereof after concentration.

Preferably, said wound is a burn wound.

The present invention also provides a method for wound healing and/or skin whitening, comprising: administrating the aforesaid pharmaceutical composition to an object.

Preferably, a route of said administrating is by spreading said active ingredient on a place to be treated of said object.

Preferably, said Andrographis paniculata extract of said pharmaceutical composition is an ethanol extract.

Preferably, said Andrographis paniculata extract of said pharmaceutical composition is made by an extraction process; wherein said extraction process comprises the following steps: obtaining an Andrographis paniculata plant; soaking said Andrographis paniculata with an ethanol to obtain a mixture; concentrating said mixture to form said extract.

Preferably, said soaking is achieved by putting said Andrographis paniculata plant into a bath of said ethanol.

Preferably, soaking is performed for 84 to 96 hours. Preferably, said soaking is performed at 20 to 25° C.

Preferably, said mixture is filtered before being concentrated.

Preferably, said concentrating is achieved by reduced pressure.

Preferably, said extraction process further comprises the step of dissolving said extract in DMSO, absolute ethanol, anhydrous ethanol, or a combination thereof after concentration.

Preferably, said wound is a burn wound.

In light of the foregoing, the present invention provides a pharmaceutical composition comprising an Andrographis paniculata extract, Andrographolide, or a combination thereof as active ingredient. The present invention also teaches to use the aforesaid pharmaceutical composition in wound healing (particular in burn wound healing) and skin whitening. In other words, the present invention provides novel pharmaceutical applications of Andrographis paniculata.

### BRIEF DESCRIPTION OF THE DRAWINGS

FIG. 1 shows the result of cell viability assay for HaCaT cells in Example 2 of the instant specification. HaCaT cells (1×10⁴ cells) were seeded in 96 well plates for overnight. Cells were treated with various concentrations of (A) A. paniculata extract and (B) Andrographolide (1, 5, 10 or 50 μg/ml) for additional 24 or 48 h. The cell viability was analyzed by MTT assay. The results of OD value were measured at 570 nm by ELISA reader. Data are normalized to normal control and represented as mean±SEM in triplicate determinations. *, p<0.05; **, p<0.01; ***, p<0.001.

FIG. 2 shows the result of cell viability assay for CCD-966SK cells is in Example 2 of the instant specification. CCD-966SK cells (1×10⁴ cells) were seeded in 96 well plates for overnight. Cells were treated with various concentrations of (A) A. paniculata extract and (B) Andrographolide (1, 5, 10 or 50 μg/ml) for additional 24 or 48 h. The cell viability was analyzed by MTT assay. The results of OD value were measured at 570 nm by ELISA reader. Data are normalized to normal control and represented as mean±SEM in triplicate determinations. *, p<0.05; ***, p<0.001.

FIG. 3 shows a series of pictures of the mice model in wound healing assay. Mice were anesthetized by pentobarbital and removed the hair of back. The special stainless steel cylinder tool (1 cm in diameter, 0.785 cm² in area) was heated for 20 sec and attached on back skin of mice for 2 sec. A. paniculata (0.1 mg/ml), Andrographolide (0.1 mg/ml), and Silver sulfadiazine (10 mg/g of body weight) were applied everyday for treatment. The area of injured skin was measured every two days until no scar formation.

FIG. 4 shows the pictures of Histopathology staining of Example 4. The healed skin in Example 3 was removed and sectioned for tissue staining. Formaldehyde-fixed, paraffin-embedded skin samples were sliced and subjected to hematoxylin and eosin (H&E) staining.

FIGS. 5A and 5B show the results of an Andrographolide treatment.

### DETAILED DESCRIPTION

The pharmaceutical potentials of A. paniculata have been reported; however, there is no any information about A. paniculata's ability in wound healing and skin whitening.

The term “wound” used herein is referred to as an injury on a skin; wherein the epidermal layer and/or the dermis layer are at least partially destroyed at the site concerned. More specifically, the term “wound” used is herein is referred to as a burn wound including 1^{st} grade, 2^{nd} grade, and/or 3^{rd} grade of burn wound. The term “wound healing” is referred to a process composed of inflammation, granulation tissue proliferation, and epidermal cell regeneration and remodeling (Takayama & Aoki, 2012).

The term “skin whitening” is referred to a process of a change in color of a skin; wherein the chromaticity of the skin concerned becomes lighter than before. The chromaticity of a skin is known to be mainly related to the expression level of melanin.

One aspect of the present invention provides a pharmaceutical composition, which comprises 0.01 to 1 wt % of an active ingredient, and 90 to 99.99 wt % of a pharmaceutical acceptable carrier. Said active ingredient is an Andrographis paniculata extract, Andrographolide, or a combination thereof. Said pharmaceutical composition may also include other pharmaceutically acceptable additives, such as, fillers, diluents, stabilizers, flavors, antioxidants, or a combination thereof. The present invention proves in the following experiments that said pharmaceutical composition have the efficacy in wound healing and/or skin whitening.

Preferably, a route of administrating the present pharmaceutical composition is by spreading said pharmaceutical composition on a place to be treated of said object. Said spreading is achieved by hand or a tool (preferably, a sterile tool). It is favorable to make the pharmaceutical composition of the present invention in a formulation of a solution, a suspension, an emulsion, an ointment because those types of formulations would be convenient for spreading.

Another aspect of the present invention provides a method for wound healing and/or skin whitening, which comprises: administrating the pharmaceutical composition of the present invention to an object. Said administrating, as set forth above, is to spread said pharmaceutical composition on a place to be treated of said object by hand or a tool.

In a preferable embodiment of the present invention, said pharmaceutical acceptable carrier is, but not limited to, water, alcohol, glycerol, chitosan, alginate, chondroitin, Vitamin E, Vitamin A, mineral oil, dimethyl sulfoxide (DMSO), or a combination thereof.

Preferable, said Andrographis paniculata extract is an alcohol extract; more preferable, is an ethanol extract. In a preferable embodiment, said Andrographis paniculata extract is prepared by the following steps: obtaining an Andrographis paniculata plant; soaking said Andrographis paniculata with an ethanol to obtain a mixture; concentrating said mixture to form said extract.

In an alternative embodiment, said Andrographis paniculata plant is a whole plant; preferable, is the aerial parts of a plant. Preferable, said ethanol is of a concentration of 90 to 95% (v/v). In a preferable embodiment, said soaking is achieved by putting said Andrographis paniculata into a bath of said ethanol.

In a preferable embodiment, soaking is performed for 84 to 96 hours. Preferably, said soaking is performed at 20 to 25° C. Alternatively, said mixture is filtered with any known filtration method in the art before being concentrated. The concentration can be achieved by any manner known in the art, for instance, but not limit to: by reduced pressure.

The following embodiments are recited for further explaining the advantages of the present invention but not for limiting the claim scope of the present invention. The following experiment results are presented as means±SEM or means±SD. Data were analyzed with one-way ANOVA Turkey test for multiple-group comparisons. The p values less than 0.05 were considered statistically significant.

### Example 1

### Preparation of Andrographis paniculata Extract of the Present Invention

Andrographis paniculata was collected from Ping Tung County, Taiwan in March 2011, and a voucher specimen was deposited in the Department of Food Science & Technology, Tajen University, Pingtung, Taiwan. The air-dried whole plant (1 kg) were cut into small pieces and soaked with ethanol (10 L×2) at room temperature for 96 hours, then filtered by filter-press filtration. The combined filtrates were concentrated and the solvent was removed under reduced pressure to afford the A. paniculata extract (130 g). The extract was dissolved in DMSO.

### Example 2

### Examining the Effect of A. paniculata Extract and Andrographolide Treatment on Cell Viability

In this example, the effect of treating A. paniculata extract or Andrographolide on cell viability was examined by MTT assay.

### [Cell Line]

HaCaT cell is an immortal human keratinocyte line (CLS order no. 300493) and A375 cell is a human melanoma cell line (ATCC, CRL-1619) that isolated from malignant melanoma. Both cells were cultured in Dulbecco's Modified Eagle Medium (DMEM; Gibco, Carlsbad, Calif., USA) with 10% fetal bovine serum (FBS; Gibco), and 1% Penicillin/Streptomycin (Biowest) in culture incubator at 37° C., 5% CO₂. to CCD-966SK is a human skin fibroblast (ATCC, CRL-1881) cell line that taken from the breast of patient with invasive ductal carcinoma and cultured in Minimum Essential Medium (MEM; Gibco) with 10% FBS, 1% sodium pyruvate (Biowest), and 1% Penicillin/Streptomycin (Biowest) in culture incubator at 37° C., 5% CO₂.

### [MTT Assay]

The 3-(4,5-dimethylthiazol-2-yl)-2,5-diphenyltetrazolium bromide (MTT, Invitrogen) is a colorimetric based assay that is performed to analyze the proliferation of cells. Cells (1×10⁴ cells) were seeded in 96 well plates for overnight. Cells were treated with various concentrations of A. paniculata extract and Andrographolide (1, 5, 10 or 50 μg/ml) for additional 24 or 48 h, after incubation 20 μl (5 mg/ml) of MTT solution (M6494; Invitrogen, Grand Island, N.Y., USA) was added per well and further incubated for 4 h. The media was removed, and formazan was solubilized by adding 100 μl/well of DMSO (Sigma-Aldrich) and absorbance was measured at 570 nm using a microplate reader (Thermo Labsystems, Opsys MR, USA). Percentage of proliferation ability was estimated by comparing with untreated control cells.

### [Results]

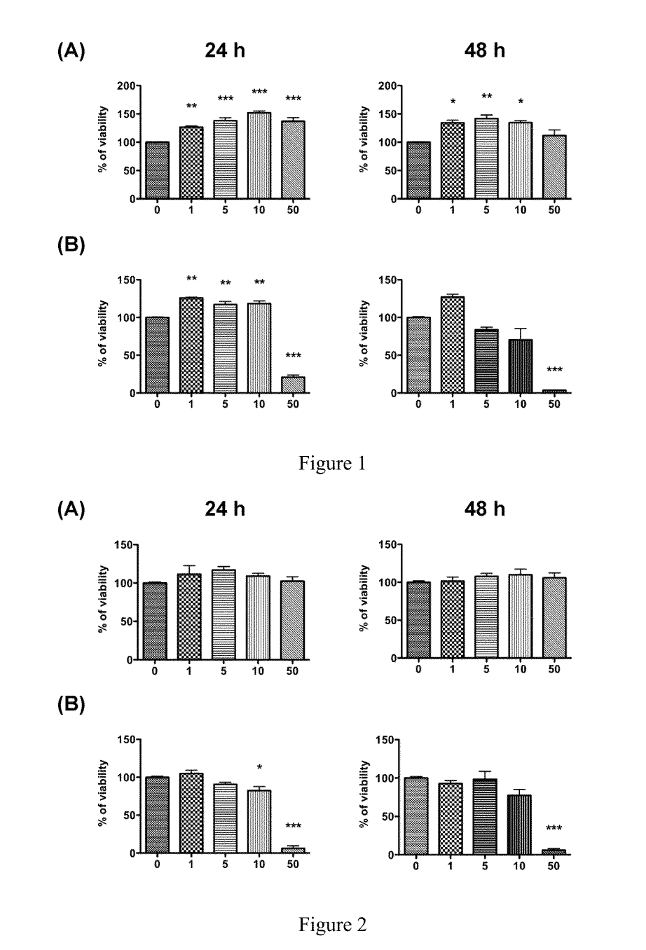

The results showed that cell viability of HaCaT cells were significantly increased 24 and 48 h after A. paniculata extract treatment (FIG. 1A). Cell viability of HaCaT cells was significantly decreased 24 and 48 h after 50 μg/ml Andrographolide treatment (FIG. 1B). Moreover, low doses (1, 5, and 10 μg/ml) Andrographolide treatment for 24 h significantly enhanced cell viability of HaCaT cells. Cell viability of CCD-966SK cells was not affected after difference doses of A. paniculata extract treatment (FIG. 2A). However, Cell viability of CCD-966SK cells was significantly decreased 24 and 48 h after 50 μg/ml Andrographolide to treatment (FIG. 2B). According to these results, high dose (50 μg/ml) Andrographolide treatment may affect cell viability of HaCaT and CCD-966SK cells in in vitro cell culture condition.

### Example 3

### Wound Healing Efficacy of A. paniculata Extract and Andrographolide Treatment

The efficacy of A. paniculata extract and Andrographolide treatment in wound healing was examined in the example.

### [Animals]

C57BL/6 mice were obtained from National Laboratory Animal Center of National Science Council, Taipei, Taiwan and maintained in the Animal Center of the National Dong Hwa University, Hualien, Taiwan. These mice were used between 8 to 10 weeks of age and age-matched for each experiment. Animal care and handling was confirmed to the Guide for the Care and Use of Laboratory Animals.

### [In Vivo Drug Preparation]

A cream matrix containing 1 g polyethylene glycol/poly propylene glycol/poly ethylene glycol (PEG-PPG-PEG; MW=5800; Sigma), 2 ml polyethylene glycol-400 (PEG-400; FCW®, Taiwan), and 3 ml polyethylene glycol-1500 (PEG-1500; FCW®, Taiwan) was formed. The solution of A. paniculata extract and Andrographolide was added into cream matrix and completely mixed (for both of them, the final concentration is 0.1 mg/ml). Silver sulfadiazine (1%; Sinphar, I-lan, Taiwan) was employed as a positive control.

### [Animal Skin Injury Model]

Mice were anesthetized by pentobarbital (50 mg/kg) and removed the hair of back. A stainless steel cylinder tool (1 cm in diameter, 0.785 cm² in area) was heated for 20 sec and attached on back skin of mice for 2 sec. The cream matrix of A. paniculata extract (0.1 mg/ml), Andrographolide (0.1 mg/ml) and Silver sulfadiazine (1%; as a positive control) was prepared as recited in the preceding paragraph and spread on the burned site of the skin once/day. The wound area was measured on days 1, 3, 5, 7, 9, 11, 13, 15, 17, 19, and 21 after drug treatment.

[Results]

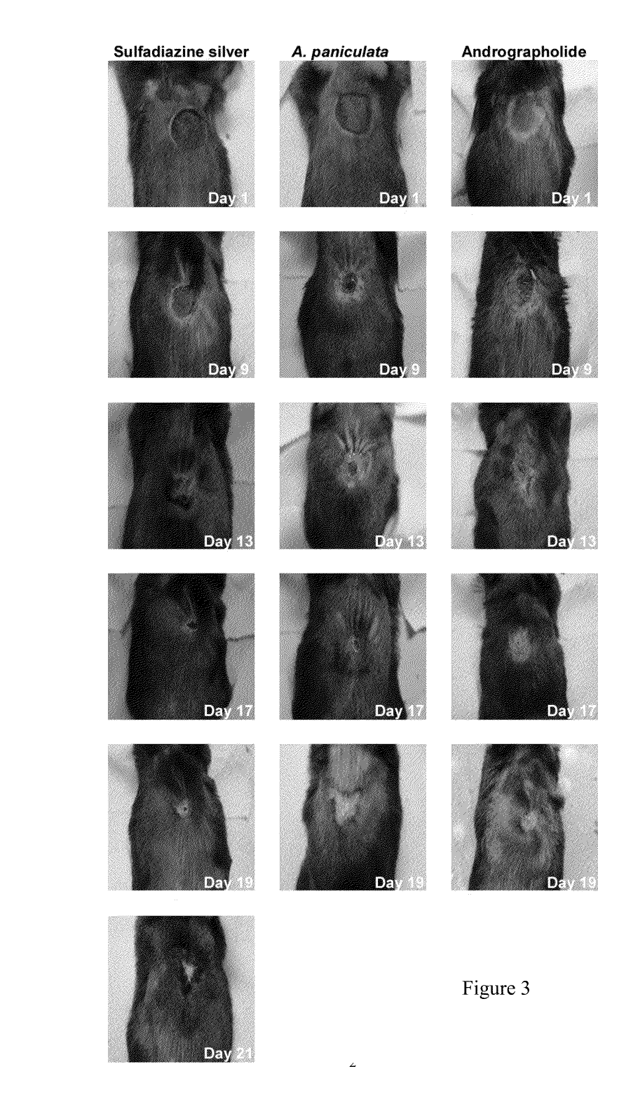

The data showed that A. paniculata extract and Andrographolide treatment enhanced wound healing rate two days earlier than that of silver sulfadiazine-treated mice (FIG. 3). The wound healing efficacy was similar between A. paniculata extract and Andrographolide. The exact values of wound area were listed on Table 1. Moreover, it is noted that the treating amount of A. paniculata extract and Andrographolide were significantly lower than that of silver sulfadiazine. According to these results, A. paniculata extract and Andrographolide shows better efficacy in wound healing then the conventional drug.

TABLE 1
Wound Area of the wound healing experiment of Example 3

| | | A. paniculata | |
|---|---|---|---|
| | Sulfadiazine silver | Extract | Andrographolide |
| DAY 1 | 115.73 ± 15.09 | 102.42 ± 5.86 | 133.57 ± 28.46 |
| DAY 3 | 111.04 ± 6.47 | 92.92 ± 13.19 | 111.94 ± 24.09 |
| DAY 5 | 85.88 ± 2.20 | 85.99 ± 21.11 | 85.15 ± 13.35 |
| DAY 7 | 58.70 ± 0.10 | 61.1 ± 26.22 | 48.43 ± 5.91 |
| DAY 9 | 36.45 ± 5.60 | 43.92 ± 26.29 | 28.22 ± 9.40 |
| DAY 11 | 23.47 ± 10.71 | 17.43 ± 17.57 | 16.46 ± 5.43 |
| DAY 13 | 12.96 ± 0.67 | 12.57 ± 12.56 | 11.62 ± 4.49 |
| DAY 15 | 8.12 ± 2.49 | 3.55 ± 3.04 | 6.94 ± 5.04 |
| DAY 17 | 3.46 ± 1.67 | 0.8635 ± 1.50 | 4.12 ± 3.96 |
| DAY 19 | 3.10 ± 2.70 | 0 | 0 |
| DAY 21 | 0 | 0 | 0 |

Note:
Data are presented as mean ± SD, unit: mm².

### Example 4

### Histopathology Examination for Skin Recovery Under A. paniculata Extract and Andrographolide Treatment

The above example 3 has shown that the both of A. paniculata extract and Andrographolide have good efficacy in wound healing. In this example, the skin recovery was examined by haematoxylin and eosin staining and Sirius red staining.

### [Histopathology]

Skin tissues were fixed with 4% formaldehyde and prepared as 10 μm thick paraffin sections. Paraffin sections were deparafinization with xylene for 5 min twice and rehydration with 99%, 95% and 75% of EtOH for 5 min twice of each. For histopathological examination, sections were stained with haematoxylin and eosin stain (H&E) for routine histology and with Sirius red for collagen.

The paraffin embedded and H&E stain steps were accomplished in Department of Physiology at Kaohsiung Medical University. For the Sirius red stain experiment, slides were stained nuclei with haematoxylin for 8 min, and then washed the slides for 10 min in running tap water. Sections were stained in picro-sirius red for 1 h (aqueous solution of saturated picric acid containing 0.1% Direct red 80 (Sigma)) at room temperature, and then washed twice with acidified water (0.5% glacial acetic acid) before dehydration with absolute anhydrous EtOH. Finally, the slides were washed with xylene and fixed in a permanent mounting medium (VectaMount™, VECTOR, Burlingame, Calif., USA).

### [Results]

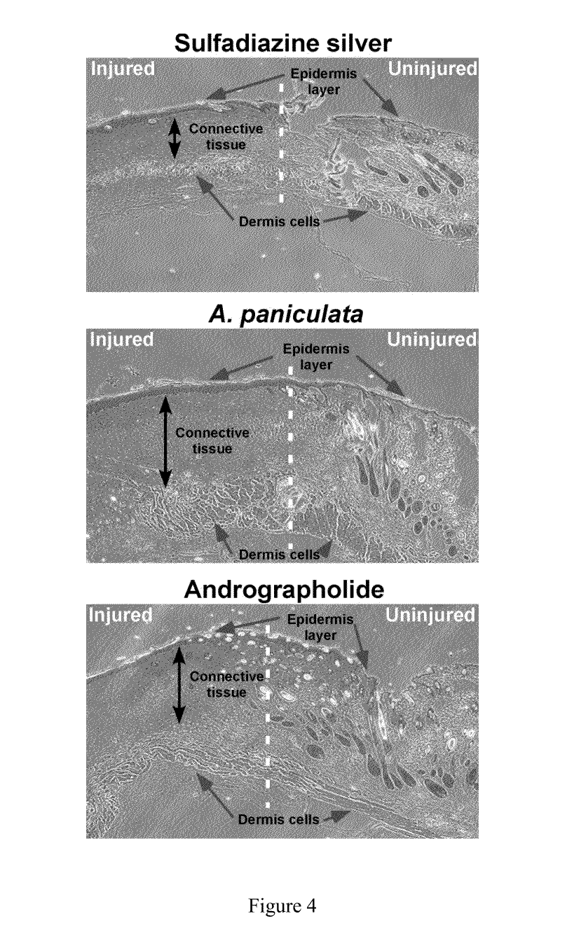

The healed skin in the above Example 3 was removed and sectioned is for tissue staining (FIG. 4). The epidermis of the healed tissue from sulfadiazine silver-treated skin was thicker than that of uninjured tissue. The dermis layer of healed tissue was replaced and filled with connective tissue. But the dermis cells near to the subcutaneous tissue were still maintained integrity. Whereas, after A. paniculata extract treatment showed that the epidermis of the healed tissue was thicker than that of uninjured tissue, and dermis layer was filled with connective tissue. Similar results were also observed in Andrographolide-treated skin tissue. Taken together, A. paniculata extract and Andrographolide exhibited similar wound healing efficacy, enhanced epidermis layer formation, compared to that of sulfadiazine silver. The result consists with the result in Example 4 shows that the present A. paniculata extract and Andrographolide treatment exhibit better efficacy than the conventional drug.

### Example 5

### The Effect of A. paniculata Extract and Andrographolide Treatment in Melanin Expression Level

Pigment incontinence in the newly-healed skin is an annoying issue. Melanin accumulation is the main reason of pigment incontinence. Therefore, the effect of A. paniculata extract and Andrographolide treatment in melanin expression level was examined in the example. A375 cell was used in this example and the cell viability was also examined based on the protocol in the aforesaid Example 2.

### [Melanin Assay]

Cells (1×10⁵ cells) were seeded in 12 well plates and treated with drugs for 24 or 48 hr. Plates were washed with PBS and added 0.5 ml of 1N NaOH, and heating at 100° C. for 1 h. Plates were then centrifuged at 12,000 rpm for 30 min at 25° C. Supernatants (100 μl/well) were added into 96 well plates, and the secretion of melanin was determined by measuring the absorbance at 405 nm using microplate reader (Thermo Labsystems).

### [Results]

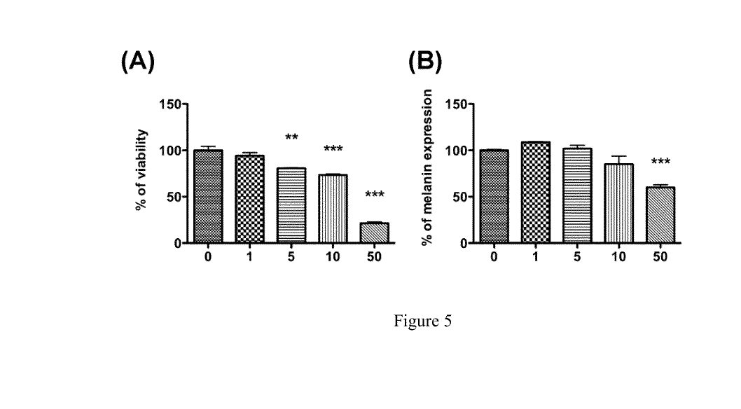

The cell viability of A375 cell was significantly inhibited after Andrographolide treatment and showed a dose-dependent manner (FIG. 5A). After normalization with cell number, the results shows that the expression level of melanin, the most abundant protein in A375 cells, was also significantly inhibited with a dose-dependent manner after Andrographolide treatment (FIG. 5B). Taken together, Andrographolide may exhibits potential for skin whitening through down regulation of melanin formation.

### REFERENCE

Breitkreutz D, Mirancea N, Nischt R (2009) Basement membranes in skin: unique matrix structures with diverse functions? Histochemistry and cell biology 132: 1-10
Caceres D D, Hancke J L, Burgos R A, Sandberg F, Wikman G K (1999) Use of visual analogue scale measurements (VAS) to assess the effectiveness of standardized Andrographis paniculata extract SHA-10 in reducing the symptoms of common cold. A randomized double blind-placebo study. Phytomedicine: international journal of phytotherapy and phytopharmacology 6: 217-223
Eming S A, Krieg T, Davidson J M (2007) Inflammation in wound repair: molecular and cellular mechanisms. The Journal of investigative dermatology 127: 514-525
Gurtner G C, Werner S, Barrandon Y, Longaker M T (2008) Wound repair and regeneration. Nature 453: 314-321
Handa S S, Sharma A (1990) Hepatoprotective activity of andrographolide against galactosamine & paracetamol intoxication in rats. Indian J Med Res 92: 284-292
Jin L, Fang W, Li B, Shi G, Li X, Yang Y, Yang J, Zhang Z, Ning G (2012) Inhibitory effect of Andrographolide in 3T3-L1 adipocytes differentiation through the PPARgamma pathway. Molecular and cellular endocrinology 358: 81-87
Mogili N S, Krishnaswamy V R, Jayaraman M, Rajaram R, Venkatraman A, Korrapati P S (2012) Altered angiogenic balance in keloids: a key to therapeutic intervention. Translational research: the journal of laboratory and clinical medicine 159: 182-189
Sharma J, Gairola S, Gaur R D, Painuli R M (2012) The treatment of jaundice with medicinal plants in indigenous communities of the Sub-Himalayan region of Uttarakhand, India. J Ethnopharmacol 143: 262-291
Singer A J, Clark R A (1999) Cutaneous wound healing. N Engl J Med 341: 738-746
Spasov A A, Ostrovskij O V, Chernikov M V, Wikman G (2004) Comparative controlled study of Andrographis paniculata fixed combination, Kan Jang and an Echinacea preparation as adjuvant, in the treatment of uncomplicated respiratory disease in children. Phytotherapy research: PTR 18: 47-53
Takayama Y, Aoki R (2012) Roles of lactoferrin on skin wound healing. Biochemistry and cell biology=Biochimie et biologie cellulaire 90: 497-503
Thisoda P, Rangkadilok N, Pholphana N, Worasuttayangkurn L, Ruchirawat S, Satayavivad J (2006) Inhibitory effect of Andrographis paniculata extract and its active diterpenoids on platelet aggregation. European journal of pharmacology 553: 39-45
Tredget E E, Yang L, Delehanty M, Shankowsky H, Scott P G (2006) Polarized Th2 cytokine production in patients with hypertrophic scar following thermal injury. Journal of interferon & cytokine research: the official journal of the International Society for Interferon and Cytokine Research 26: 179-189
Yang S, Evens A M, Prachand S, Singh A T, Bhalla S, David K, Gordon L I (2010) Mitochondrial-mediated apoptosis in lymphoma cells by the diterpenoid lactone andrographolide, the active component of Andrographis paniculata. Clinical cancer research: an official journal of the American Association for Cancer Research 16: 4755-4768
Yoopan N, Thisoda P, Rangkadilok N, Sahasitiwat S, Pholphana N, Ruchirawat S, Satayavivad J (2007) Cardiovascular effects of 14-deoxy-11,12-didehydroandrographolide and Andrographis paniculata extracts. Planta medica 73: 503-511
Zaja-Milatovic S, Richmond A (2008) CXC chemokines and their receptors: a case for a significant biological role in cutaneous wound healing. Histology and histopathology 23: 1399-1407

1. A pharmaceutical composition for wound healing and/or skin whitening, comprising:
0.01 to 1 wt % of an active ingredient, comprising an Andrographis paniculata extract, Andrographolide, or a combination thereof; and
90 to 99.99 wt % of a pharmaceutical acceptable carrier.

2. The composition of claim 1, wherein said pharmaceutical acceptable carrier is water, alcohol, glycerol, chitosan, alginate, chondroitin, Vitamin E, Vitamin A, mineral oil, dimethyl sulfoxide (DMSO), or a combination thereof.

3. The composition of claim 1, wherein an administrating route of said composition via spreading said composition on a place to be treated of an object.

4. The composition of claim 1, wherein said Andrographis paniculata extract is made by the following steps:
obtaining a Andrographis paniculata plant;
soaking said Andrographis paniculata with an ethanol to obtain a mixture;
concentrating said mixture to form said extract.

5. The composition of claim 4, wherein said soaking is performed at 20 to 25° C. for 84 to 96 hours.

6. The composition of claim 4, wherein said extraction process further comprises the step of dissolving said extract in DMSO, absolute ethanol, anhydrous ethanol, or a combination thereof after concentration.

7. The composition of claim 1, wherein said wound is a burn wound.

8-18. (canceled)
